(19) United States
(12) Patent Application Publication
(10) Pub. No.: US 2005/0261569 A1
(43) Pub. Date: Nov. 24, 2005

(54) Catheter for use in mr imaging

(75) Inventors: Volkmar Schulz, Lubeck (DE); Bernhard Gleich, Hamburg (DE)

Correspondence Address: PHILIPS INTELLECTUAL PROPERTY & STANDARDS, 595 MINER ROAD, CLEVELAND, OH, 44143, US

(73) Assignee: Koninklijke Philips Electronics N.V., Eindhoven (NL)

(21) Appl. No.: 10/526,735

(22) PCT Filed: Aug. 25, 2003
(86) PCT No.: PCT/IB03/03785

§ 371 Date: Mar. 4, 2005

(30) Foreign Application Priority Data
Sep. 5, 2002 (DE) 102 40 960.9

(51) Int. Cl. A61B 5/05

(52) U.S. Cl. 600/411

(57) ABSTRACT

The invention relates to a catheter which is suitable in particular for use in MR imaging. In order to avoid undesirable heating of the tissue surrounding the catheter by the MR excitation field, the catheter in accordance with the invention comprises: a catheter sleeve (2), a hollow guide channel or lumen (3) within the catheter sleeve (2) for the introduction of a medical instrument, and two electrical conductors (4) which are enclosed by a cable sheath (5) of a dielectric material and serve to transmit RF signals within the catheter envelope (2), the dielectric material having a relative permittivity (ε [symbol] r?) smaller than 4, the diameter of the electrical conductors (4) being between 5 and 50 μm, notably between 10 and 30 μm, and the distance between the electrical conductors (4) being smaller than 300 μm, in particular smaller than 200 μm.

The invention relates to a catheter which is suitable especially for use in magnetic resonance imaging (MR imaging), as well as to an MR device for forming MR images of an object to be examined, in particular for intravascular interventional MR imaging.

A catheter for use in MR imaging is known from U.S. Pat. No. 5,792,055. The catheter therein is formed by a coaxial cable which serves as an antenna. As a result, the catheter can be localized during an intravascular intervention so as to be imaged in MR images. It is a drawback, however, that in response to the RF excitation of the spins the tissue surrounding the coaxial cable is heated due to the standing waves arising around the cable (common mode resonance). This could damage the surrounding tissue.

Therefore, it is an object of the invention to provide a catheter, in particular for use in MR devices, as well as a corresponding MR device in which the described undesirable heating of the tissue surrounding the catheter is avoided.

This object is achieved by means of a catheter as disclosed in claim 1 which comprises:
a catheter sleeve (2),
a hollow guide channel or lumen (3) within the catheter sleeve (2) for receiving a medical instrument, and
two electrical conductors (4) which are enclosed by a cable sheath (5) of a dielectric material and serve for the transmission of RF signals within the catheter sleeve (2), the dielectric material having a relative permittivity (εᵣ) which is smaller than 4, the diameter of the electrical conductors (4) being between 5 and 50 μm, notably between 10 and 30 μm, and the distance between the electrical conductors (4) being smaller than 300 μ, in particular smaller than 200 μm.

The object of the invention is also achieved by means of an MR device as claimed in claim 6 which includes:
a main field magnet system (16) for generating a homogeneous, steady main magnetic field,
a gradient coil system (17, 18, 19) for generating magnetic gradient fields,
an RF coil system (14) for exciting an examination zone,
a receiving coil system (14, 12) for receiving MR signals from the examination zone,
a catheter (1) as claimed in claim 1 for introducing a medical instrument into the object (10) to be examined, notably comprising an active coil (4, 5) which is arranged on or in the catheter (1) for the purpose of catheter localization, local excitation of the examination zone and/or local reception of MR signals, and
a control unit (23) for controlling the MR device.

The invention is based on the idea to configure the catheter in such a manner that no resonance can occur up to the MR frequency used. To this end, in accordance with the invention there is provided a cable which comprises two electrical conductors which are enclosed by a cable sheath of a dielectric material, the cable being constructed in such a manner that it has a low shortening factor. In this context the shortening factor is defined as the square root of the product of the relative permittivity (εᵣ) and the relative permeability (μᵣ), the shortening of the wavelength used resulting from the fact that the electromagnetic wave does not propagate in vacuum but in a medium having a relative permittivity and/or relative permeability larger than 1. When a shortening factor is chosen in this manner, the common mode resonance of the cable is shifted beyond the MR frequency.

Moreover, in accordance with the invention it is arranged to utilize a miniaturized pair of cables where the individual conductors have only a small diameter and are situated at a small distance from one another. In order to achieve an as small as possible shortening factor also during the intervention, the diameters of the conductors should be as small as possible; however, they should not be too small, as otherwise large signal losses will occur. Therefore, the indicated orders of magnitude represent a suitable compromise.

Advantageous embodiments of the catheter in accordance with the invention are disclosed in the dependent claims. In a preferred embodiment the dielectric material has a relative permittivity which is smaller than 2.3, notably smaller than 1.5. For example, polytetrafluoroethylene (PTFE), having a relative permittivity of approximately 2.3, could be used as the dielectric material.

Alternatively, in particular aerated synthetic materials are suitable for use as a dielectric material for the cable sheath, because the relative permittivity of such materials is nearly 1. An example in this respect is, for example, the material FP301040 or FP301020 (marketed by Good Fellow). Shortening factors as small as 1.2 can thus be achieved, the choice of the suitable dielectric material also being dependent on the field strength of the main field magnets of the MR device used.

In conformity with a further embodiment of the invention, the two electrical conductors are also arranged to conduct a direct voltage for the voltage supply of a medical instrument arranged on or in the catheter. An example in this respect is an active coil in conformity with a further embodiment which is arranged at the tip of the catheter and can act for catheter localization during an intervention or also for MR signal acquisition in its direct vicinity.

The invention can in principle be used in all MR devices in which especially intravascular interventions have to be carried out, in particular in MR devices with a field strength of up to 2 Tesla, that is, for typical patient sizes; when only small catheter lengths are required, for example, in the case of small children, examinations can also be performed with greater field strengths. In a 1.5 Tesla system catheters can be used with a length of up to 1.6 m. The catheter in accordance with the invention thus constitutes an economical solution which can be readily implemented and whereby the undesirable heating of the tissue surrounding the catheter by the excitation field for the spins is avoided.

The invention will be described in detail hereinafter with reference to the drawings. Therein:

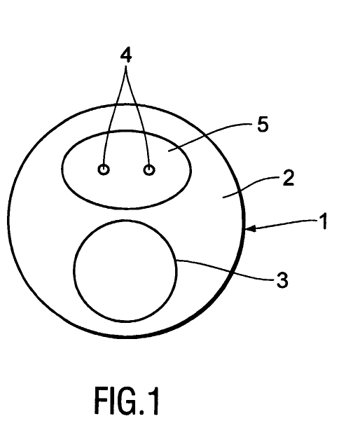

FIG. 1 is a cross-sectional view of a catheter in accordance with the invention, and

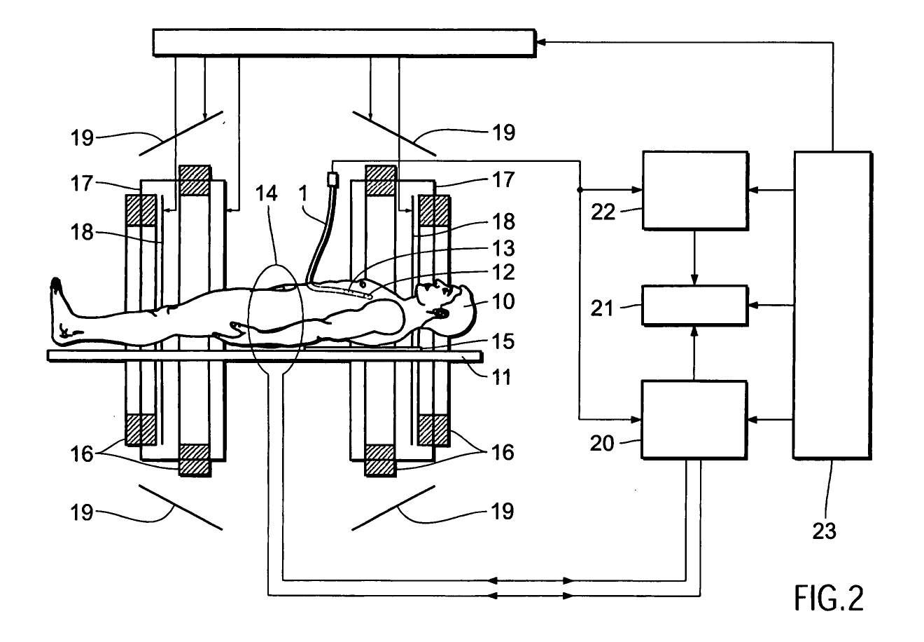

FIG. 2 is a simplified representation of an MR device in accordance with the invention which is provided with a catheter of this kind.

FIG. 1 is a cross-sectional view of a catheter 1 in accordance with the invention. It consists of a catheter sleeve 2 which consists, for example, of a flexible synthetic material. Inside the catheter sleeve 2 there is formed a guide channel (lumen) 3 wherethrough one or more medical instruments can be introduced into the object to be examined, for example, the body of a patient. Furthermore, inside the catheter sleeve 2 there is provided a cable sheath 5 in which two electrical conductors 4 extend substantially parallel to one another so that they are completely enclosed by the cable sheath 5. The cable sheath 5 consists of a dielectric material having a dielectric number (permittivity) εᵣ which is smaller than 4, preferably smaller than 2.3. The diameter of the electrical conductors 4 preferably is in the range of between 10 and 30 μm, for example, 15 μm, and the distance between the conductors 4 preferably is smaller than 200 μm, for example, 50 μm.

The described configuration results in a small shortening factor and the lowest resonance frequency of the cable is thus shifted to a range which does not correspond to the MR frequency used and which does not change due to the presence of the tissue. The shortening factor has an effect on the resonance frequency in a sense that the resonance frequency of an antenna is in principle inversely proportional to the shortening factor. The use of a small diameter for the electrical conductors ensures that the electromagnetic energy is preferably stored in the vicinity of the conductor and hence less energy can be dissipated in the proximity, so that heating of the proximity of the catheter is also avoided.

Two conductors are provided so as to transmit signals in the so-called differential mode. In addition, a direct voltage which does not interfere with the MR signal in the RF range can also be conducted.

A suitable dielectric material for the cable sheath 5 is, for example, polytetrafluoroethylene which has a relative permitivity of from approximately 2.2 to 2.3. Preferably, use is made of an aerated, spongy synthetic material, for example, FP301040 or FP301020 (as marketed by Good Fellow), because such materials have a relative permittivity near 1.

FIG. 2 is a diagrammatic representation of an MR device in accordance with the invention in which the catheter in accordance with the invention can be used. A patient 10 is arranged on a patient table 11 in order to carry out an intravascular intervention. A catheter 1 in accordance with the invention has been introduced into a main artery of the patient 10 in order to perform a treatment on the coronary arteries; it has been advanced as far as the coronary arteries by a physician. At its end which is introduced into the patient 10 the catheter is provided with an image acquisition device 12 and a localization device 13. The image acquisition device 12 may be, for example, a microcoil which is capable of receiving MR signals from its vicinity after excitation by means of an external excitation coil 14, said MR signals providing image information on the vicinity of the microcoil 4. By way of example, the localization device 13 is constructed as a magnetic field sensor which cooperates with a coil system 15 arranged underneath the patient 10. Using the signals emitted by the individual coils of the coil array 15, the position of the magnetic field sensor, and hence the position of the end zone of the catheter 1, can be determined on the basis of the signals received by the magnetic field sensor. The described image acquisition by means of the microcoil 12 and the localization by means of the magnetic field sensor 13 are known per se and, therefore, will not be elaborated herein.

The MR device also includes a main field magnet system 16 which comprises a plurality of main field magnets which generate a steady, uniform magnetic field in the longitudinal direction of the patient 10. In order to generate magnetic gradient fields there is provided a gradient coil system which comprises a plurality of gradient coils 17, 18, 19.

Furthermore, an RF coil system 14 is provided so as to generate RF excitation pulses and to pick up MR signals from the excited examination zone.

For the processing of the signals received by the microcoil 12 or for the control of the microcoil 12 and the excitation coil 14 there is provided an image processing and control device 20 which converts the measured signals into image information which is applied to a data processing device 21. A position processing and control unit 22 is provided for the processing of the signals picked up by the magnetic field sensor 13 and for controlling the magnetic field sensor and the coil array 15, which unit 22 converts the measured signals into position data which is applied to the data processing device 21. The control of said coils and units is performed by a control unit 23. The evaluation and reproduction of said signals as well as the operation of such an MR device are also known per se and, therefore, will not be elaborated either.

The catheter in accordance with the invention, which can be manufactured simply and economically, effectively prevents the heating of the part of the tissue of the patient 10 which surrounds the catheter. The catheter can be used for various applications in MR imaging.

1. A catheter for use in MR imaging and which includes
a catheter sleeve,
a hollow guide channel or lumen within the catheter sleeve for receiving a medical instrument, and
two electrical conductors which are enclosed by a cable sheath of a dielectric material and serve for the transmission of RF signals within the catheter sleeve, the dielectric material having a relative permittivity (εᵣ) smaller than 4, the diameter of the electrical conductors being between 5 and 50 μm, and the distance between the electrical conductor being smaller than 300 μm.

2. A catheter as claimed in claim 1, wherein the dielectric material has a relative permitivity which is smaller than 2.3.

3. A catheter as claimed in claim 1, wherein the dielectric material is an aerated synthetic material.

4. A catheter as claimed in claim 1, wherein the two electrical conductors are also arranged to conduct a direct voltage to the voltage supply of a medical instrument arranged on or in the catheter.

5. A catheter as claimed in claim 1, wherein it includes means for catheter localization during an intervention, said means fro catheter localization including at least one active coil which is arranged on or in the catheter.

6. An MR device for forming MR images of an object to be examined, which device includes:
a main field magnet system for generating a homogeneous steady main magnetic field,
a gradient coil system for generating magnetic gradient fields,
an RF coil system for the exciting an examination zone,
a receiving coil system for receiving MR signals from the examination zone,
a catheter for introducing a medical instrument into the object to be examined, said catheter comprising:
an active coil which is arranged on or in the catheter for the purpose of catheter localization, local excitation of the examination zone and/or local reception of MR signals; two electrical conductors which are enclosed by a cable sheath of a dielectric material and serve for the transmission of RF signals within the catheter sleeve, the dielectric material having a relative permittivity (εᵣ) smaller than 4, the diameter of the electrical conductors being between 5 and 50 μm and the distance between the electrical conductor being smaller than 300 μm; and
a control unit for controlling the MR device.
